(19) United States
(12) Patent Application Publication
(10) Pub. No.: US 2012/0093779 A1
(43) Pub. Date: Apr. 19, 2012

(54) COMPLEX MICROBIAL PREPARATION FOR TREATING DIABETES AND PREPARATIVE METHOD AND USE THEREOF

(76) Inventor: Liping Wang, Beijing (CN)

(21) Appl. No.: 13/257,368

(22) PCT Filed: Nov. 3, 2010
(86) PCT No.: PCT/CN2010/001769

§ 371 Date: Sep. 19, 2011

(30) Foreign Application Priority Data
Nov. 25, 2009 (CN) 200910238778.5

(51) Int. Cl. A61K 35/74; A61P 3/10

(52) U.S. Cl. 424/93.3

(57) ABSTRACT

A complex microbial preparation comprises (in vol. %): Photosynthetic bacterium (5-15%), Bacillus (10-20%), Yeasts (20-35%), Lactobacillus (30-45%) and Actinomycetes (3-10%). The preparative method of the complex microbial preparation involves first and second level complex fermentations. The first level complex fermentation involves: (a) separately inoculating the above-mentioned bacteria on first level culture media and culturing to obtain first level bacteria, (b) separately inoculating the obtained first level bacteria on second level culture media and culturing to obtain fermenting bacterial liquids, (c) mixing the five obtained fermenting bacterial liquids to obtain first level compound bacteria. The second level complex fermentation involves: adding sterile water, Lactobacillus plantarum, Lactobacillus acidophilus, Saccharomyces cerevisiae and brown sugar to the obtained first level compound bacteria, mixing uniformly in a same fermentor, and culturing to obtain second level complex microbial preparation. The complex microbial preparation is used in treatment for diabetes.

### CROSS-REFERENCE TO RELATED APPLICATIONS

The present application claims priority to PCT Application PCT/CN2010/001769 filed on Nov. 3, 2010, and Chinese Patent Application No. 200910238778.5 filed on Nov. 25, 2009, the entirety of each of which are incorporated by this reference.

### BACKGROUND

1. Field of the Invention

The present invention belongs to the field of biotechnology, in particular, relates to a complex microbial preparation and preparative method thereof and its use in producing a medicament for treatment of diabetes.

2. State of the Related Art

Diabetes is a common, frequently-occurring and lifelong disease, the cause of the disease is that the body can not normally release or use insulin and lead to an improper increase of the level of glucose (a kind of monosaccharide) in blood. The investigation of the World Health Organization (WHO) shows that, at present, the population of patients with diabetes in the world is up to about 160 millions, and there are over 60 millions diabetes patients in China, which accounts for one third of the patients with diabetes in the world. According to statistics, among people with diseases caused by diabetes, people with blindness are 10-23 times, people with gangrene and amputation are 20 times, people with kidney failure are 17 times, people with coronary disease and apoplexy are 2-3 times, and people with sudden death are 3-8 times more than those in ordinary people. For this reason, the WHO appealed that it is of vital importance to treat diabetes as early as possible for reducing the risk of complications.

Drug therapy for diabetes includes insulin therapy, chemical drug therapy by oral hypoglyceimic agents and traditional Chinese medicine therapy, and current biotechnology therapy. Chinese Patent Application No. 99105621 “A drug for treating diabetes” discloses that, a drug for treating diabetes is prepared with extract of metabolic products from single microbial strains. Chinese Patent No. 00804999 “A medicament for treatment of diabetes” relates to physiologically acceptable enzyme mixtures originated from microbe or animal. In Chinese Patent No. 01130450, the “Biological hypoglycemic agents” consists of three microorganisms i.e. Actinomycetes, Lactobacillus and Yeasts, and is prepared through conventional activation, enlarged culture, fermentation, filtering, dreg elimination, disinfection, freeze drying and other steps.

For the treatment of diabetes, the chemical drug is expensive, and it can bring significant side effects and only cure the symptom, not the disease; the traditional Chinese medicine is slow to take effect and needs long time for treating; while the biotechnology therapy can overcome the drawbacks of the above two medicines. In Chinese Patent No. 99105621, “Biological hypoglycemic agent” is a single microbial strain with a sole therapeutic effect. The medicament in Chinese Patent No. 00804999 is an enzymatic preparation as an aid in the treatment of diabetes. In Chinese Patent No. 01130450, although the hypoglycemic biopreparation consists of three microorganisms i.e. Actinomycetes, Lactobacillus and Yeasts, however, it is only a simple mixture, the blending ratio of the three microbes was not defined; there are not certain amount of clinical verifications, and its clinical effect needs to be evaluated.

### SUMMARY OF THE INVENTION

The invention provides a complex microbial preparation. The complex microbial preparation according to the invention mainly comprises of a plurality of probiotics components including Photosynthetic bacterium, Bacillus, Yeasts, Lactobacillus and Actinomycetes.

In one embodiment, the above mentioned components are respectively 5-15% by volume of Photosynthetic bacterium, 10-20% by volume of Bacillus, 20-35% by volume of Yeasts, 30-45% by volume of Lactobacillus and 3-10% by volume of Actinomycetes. In another embodiment, the above mentioned components are respectively 5-10% by volume of Photosynthetic bacterium, 10-14% by volume of Bacillus, 30-35% by volume of Yeasts, 41-45% by volume of Lactobacillus and 3-8% by volume of Actinomycetes by volume. In another embodiment, the above mentioned components are respectively 10% by volume of Photosynthetic bacterium, 10% by volume of Bacillus, 30% by volume of Yeasts, 45% by volume of Lactobacillus and 5% by volume of Actinomycetes.

In yet another embodiment, the Photosynthetic bacterium is Rhodopseudomonas palustris, the Bacillus is Bacillus subtilis, the Yeasts is Saccharomyces cerevisiae, the Lactobacillus is Lactobacillus acidophilus and the Actinomycetes is Streptomyces jingyangensis.

The present invention also provides a preparative method of the complex microbial preparation. The preparative method comprises the steps of first and second level complex fermentations of Photosynthetic bacterium, Bacillus, Yeasts, Lactobacillus and Actinomycetes, respectively.

The first level complex fermentation comprises steps of:

(1) separately inoculating mother seed of the Photosynthetic bacterium, Bacillus, Yeasts, Lactobacillus and Actinomycetes on first level culture media and conducting a first level cultivation to obtain first level bacteria;

(2) separately inoculating the obtained first level bacteria of the Photosynthetic bacterium, Bacillus, Yeasts, Lactobacillus and Actinomycetes on second level culture media and conducting a second level cultivation to obtain fermenting bacterial liquids;

(3) uniformly mixing the five obtained fermenting bacterial liquids in a specific ratio to obtain first level compound bacteria.

For the first level cultivation in step (1), the Photosynthetic bacterium are cultured with 0.5-1% of inoculation amount, for example 0.5%, with a pH of 6-10 and a light intensity of 3,000-4,000 Lux, at 28-36° C. for 7-10 days; the Bacillus are cultured with 0.5-1% of inoculation amount, for example 0.5%, with a pH of 6-8, at 28-34° C. for 2-3 days; the Yeasts are cultured with 0.5-1% of inoculation amount, for example 0.5%, with a pH of 4-7, at 25-30° C. for 1-2 days; the Lactobacillus are cultured with 0.5-1% of inoculation amount, for example 0.5%, with a pH of 6-8, at 28-34° C. for 2-5 days; and the Actinomycetes are cultured with 0.5-1% of inoculation amount, for example 0.5%, with a pH of 6-8, at 27-32° C. for 5-7 days.

For the second level cultivation in step (2), the Photosynthetic bacterium are for example cultured by intermittent aerobic cultivation with 5-15% of inoculation amount, for example 10%, with a pH of 6-10 and a light intensity of 3,000-4,000 Lux, at 28-36° C. for 7-10 days; the Bacillus are for example cultured by aerobic cultivation with 5-15% of inoculation amount, for example 10%, with a pH of 6-8, at 28-32° C. for 2-5 days; the Yeasts are for example cultured by aerobic cultivation with 5-15% of inoculation amount, for example 10%, with a pH of 4-7, at 25-30° C. for 1-2 days; the Lactobacillus are for example cultured by anaerobic cultivation with 5-15% of inoculation amount, for example 10%, with a pH of 6-8, at 28-34° C. for 2-5 days; and the Actinomycetes are for example cultured by aerobic cultivation with 5-15% of inoculation amount, for example 10%, with a pH of 6-8, at 27-32° C. for 5-7 days.

The second level complex fermentation comprises steps of:

adding sterile water to the above first level compound bacteria to dilute it 2-4 times, then adding 10-20% of Lactobacillus plantarum, 10-20% of Lactobacillus acidophilus, 10-20% of Saccharomyces cerevisiae and 5-15% of brown sugar thereto, mixing uniformly in a fluid reservior, fermenting under sterile and closed condition, culturing with a pH of 5-8 at 25-35° C. for 2-3 days to obtain second level compound bacteria.

The second level compound bacterial suspension is sampled and detected, results show: it has liquid appearance, uniform color and special odor produced by microorganism fermentation; pH value is directly measured with acidity meter and normal pH value is ranging from 5 to 8; the viable bacteria count and undesired bacteria count are measured by plate counting, a qualified product has the total viable bacteria count of more than 5×Ig and the aerobic bacteria count of 5% or less.

Still another embodiment of the invention provides use of the complex microbial preparation in producing a medicament for treatment of diabetes. The complex microbial preparation according to the invention was administered to patients with diabetes three times a day with 20 ml each time. 15 days was taken as a course of treatment, and the patients recovered to normal blood glucose level after 3 courses of treatment with an efficiency of over 80%.

Certain aspects of the invention are as follows:

1. Among the components of the complex microbial preparation according to the invention, Photosynthetic bacterium and Bacillus have the function of maintaining micro-ecological balance; Yeasts contains a plurality of proteins, vitamin B, nucleic acids and mineral substances capable of regulating metabolic functions of human body; Lactobacillus exhibits many physiological functions in animal body, e.g. reducing the level of serum cholesterol, controlling endotoxin, inhibiting the growth of intestinal spoilage bacteria, and improving immune function of organisms; Actinomycetes can produce antibiotic substances;

2. A plurality of probiotics including Photosynthetic bacterium, Bacillus, Yeasts, Lactobacillus and Actinomycetes in a specific ratio, two level complex fermentations are conducted to obtain a complex microbial preparation according to the invention.

3. Utilizing the synergetic function of a plurality of beneficial bio-regulatory factors, amino acids, antibiotic substances, various complex microbial active proteins and active enzymes which are produced after fermentation, the complex microbial preparation according to the invention is used to produce medicaments for treating diabetes. The complex microbial preparation according to the invention can rapidly regulate metabolic function of human body, repair and recombine pancreatic islet cell mass, transform the redundant glucose in the blood, reduce the burden of impaired pancreatic β cells, gradually recover the function of the impaired pancreas islets, improve microcirculation perfusion, maintain steady state of the inter environment, keep the balance of organism, correct metabolic disorder and unbalance, fundamentally eliminate diabetes and the complications, improve life quality of diabetic patients, get rid of the clinical disadvantages of taking western medicine and strictly control the diet diabetes in a long term for the diabetic patients. The complex microbial preparation according to the invention is cost effective and rapid to take effect with a good taste, while applying the preparation, there is no need to control diet and both the symptoms and root cause can be treated.

The main efficacy of the complex microbial preparation according to the invention is as follows:

1. Repairing and recombining pancreatic islet cell mass, transforming the redundant glucose in the blood, reducing the burden of impaired pancreatic β cells, activating the repair factors, recovering the function of the impaired pancreas islets, inhibiting the occurrence of complications, improving self-immunity, enhancing circulation of micro-ecosystem, finally completely recovering the function of the pancreas islets and reaching a result of recovering fully and addressing both the symptoms and root cause.

2. Overall adjusting the body function of the patients by regulating metabolism to comprehensively control the blood glucose, restoring the ability of self-regulating the blood glucose of human body by activating self-function of the patients to adjust the blood glucose to a normal level.

3. Improving microcirculation perfusion to enhance oxygen and blood supply of cells; improving blood perfusion to increase blood flow of tissues and treat and prevent diabetic micro-vascular complications and prevent fatal harm from diabetes.

4. Elevating the velocity of nerve transmission and increasing acral temperature, improving acral pruritus and prickling of diabetes, as well as preventing the occurrence of gangrene and amputation.

5. Improving sleep of the patients with neurological complication, correcting obstinate insomnia and the state of fatigue, lassitude etc.

6. Improving the diseases including diarrhea and constipation which are caused by functional disorders of the digestive system of diabetes, and hypertension caused by diabetes.

7. Maintaining steady state of the inter environment in human body, keeping balance of the organism, and correcting metabolic disorder and unbalance.

### BRIEF DESCRIPTION OF THE DRAWINGS

FIG. 1 is a flow diagram of first level complex fermentation;

FIG. 2 is a flow diagram of second level complex fermentation.

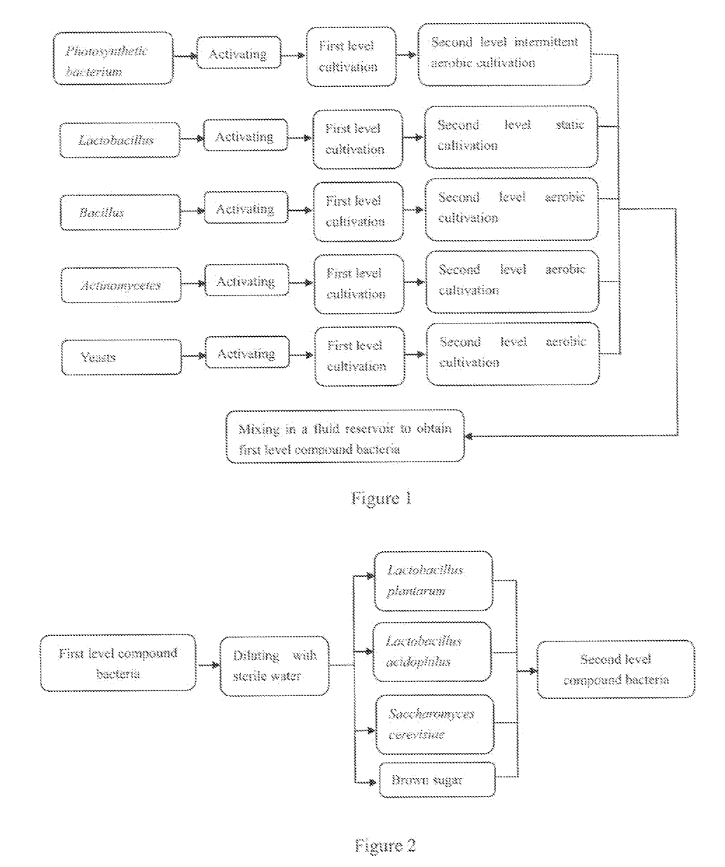

### DETAILED DESCRIPTION OF THE INVENTION

Examples are described below to further explain the contents of the invention. The scope of the invention should not be limited by the examples.

Medium for Various Bacteria

First level culture media for Rhodopseudomonas palustris: 0.2 g of •, 1.0 g of ( ), 5.0 g of, 0.5 g of, 0.2 g of NaCl and 1.5 g of peptone were dissolved in 400 ml of distilled water, the pH was adjust to 7.1 with and, then distilled water was added to adjust the volume to 1000 ml, and sterilized at 115° C. for 10 minutes; second level culture media: to 3 g of, 0.1 g of NaCl, 0.3 g of ( ) 0.2 g of, 0.5 g of, 0.3 g of, 7 ml of Ferric ammonium citrate and 1.2 g of peptone was added distilled water to make a total volume of 1000 ml, and the pH was adjusted to 7.2 with and.

First level culture media for Bacillus subtilis: to 0.3% of Beef extract, 1.0% of peptone and 0.5% of NaCl was added distilled water to make a total volume of 1000 ml, pH 7.0; second level culture media: to 1.0% of Beef extract, 1.0% of peptone and 0.5% of NaCl was added distilled water to make a total volume of 1000 ml, pH 7.0.

First level culture media for Saccharomyces cerevisiae: to 800 ml of potato juice, 20 g of glucose, 20 g of agar was added distilled water to make a total volume of 1000 ml, pH 6.0; second level culture media: to 1% of yeast extract, 2% of peptone, 2% of glucose, 0.06% of and 0.25% of was added water to make a total of 100%, pH 6.0.

First level culture media for Lactobacillus acidophilus: to 1 g of, 10 g soluble starch, 0.5 g of, 0.5 g of, 0.5 g of NaCl, 0.01 g of and 20 g of agar was added distilled water to make a total volume of 1000 ml, pH 7.2; second level culture media: to 0.8 g of, 8 g of soluble starch, 5 g of glucose, 5 g of yeast juice, 0.3 g of, 0.2 g of, 0.4 g of NaCl and 0.05 g of was added distilled water to make a total volume of 1000 ml, pH7.2.

First level culture media for Streptomyces jingyangensis: to 1 g of, 20 g of soluble starch, 0.5 g of, 0.5 g of, 0.5 g of NaCl, 0.01 g of and 20 g of agar was added distilled water to make a total volume of 1000 ml, pH 7.2; second level culture media: to 0.8 g of, 8 g of soluble starch, 5 g of glucose, 5 g of yeast juice, 0.3 g of, 0.2 g of, 0.4 g of NaCl, 0.05 g of was added distilled water to make a total volume of 1000 ml, pH7.2.

### Example 1

### Preparative Method of the Complex Microbial Preparation

First Level Complex Fermentation:

(1) 300 ml of eggplant bottle strains of Rhodopseudomonas palustris was inoculated in 60 L of the first level culture media for Rhodopseudomonas palustris, cultured at 28° C. with a pH of 6 and light intensity of 3000 Lux for 10 days to obtain first level bacteria of Rhodopseudomonas palustris;

300 ml of eggplant bottle strains of Bacillus subtilis was inoculated in 60 L of the first level culture media for Bacillus subtilis, cultured at 28° C. with a pH of 6 for 3 days to obtain first level bacteria of Bacillus subtilis;

300 ml of eggplant bottle strains of Saccharomyces cerevisiae was inoculated in 60 L of the first level culture media for Saccharomyces cerevisiae, cultured at 25° C. with a pH of 4 for 2 days to obtain first level bacteria of Saccharomyces cerevisiae;

300 ml of eggplant bottle strains of Lactobacillus acidophilus was inoculated in 60 L of the first level culture media for Lactobacillus acidophilus, cultured at 28° C. with a pH of 6 for 5 days to obtain first level bacteria of Lactobacillus acidophilus;

300 ml of eggplant bottle strains of Streptomyces jingyangensis was inoculated in 60 L of the first level culture media for Streptomyces jingyangensis, cultured at 27° C. with a pH of 6 for 7 days to obtain first level bacteria of Streptomyces jingyangensis;

(2) 60 L of the first level bacteria of Rhodopseudomonas palustris was inoculated in 600 L of second level culture media for Rhodopseudomonas palustris, cultured by intermittent aerobic cultivation at 28° C. with a pH of 6 and light intensity of 3000 Lux for 10 days to obtain fermenting bacterial suspension of Rhodopseudomonas palustris; with determination, viable count of the bacteria in the fermenting bacterial suspension is 5×-10×.

60 L of the first level bacteria of Bacillus subtilis was inoculated in 600 L of second level culture media for Bacillus subtilis, cultured by aerobic cultivation at 28° C. with a pH of 6 for 3 days to obtain fermenting bacterial suspension of Bacillus subtilis; with determination, viable count of the bacteria in the fermenting bacterial suspension is 30×-50×.

60 L of the first level bacteria of Saccharomyces cerevisiae was inoculated in 600 L of second level culture media for Saccharomyces cerevisiae, cultured by aerobic cultivation at 25° C. with a pH of 4 for 2 days to obtain fermenting bacterial suspension of Saccharomyces cerevisiae; with determination, viable count of the bacteria in the fermenting bacterial suspension is 5×-10×.

60 L of the first level bacteria of Lactobacillus acidophilus was inoculated in 600 L of second level culture media for Lactobacillus acidophilus, cultured by static anaerobic cultivation at 28° C. with a pH of 6 for 5 days to obtain fermenting bacterial suspension of Lactobacillus acidophilus; with determination, viable count of the bacteria in the fermenting bacterial suspension is 20×-30×.

60 L of the first level bacteria of Streptomyces jingyangensis was inoculated in 600 L of second level culture media for Streptomyces jingyangensis, cultured by aerobic cultivation at 27° C. with a pH of 6 for 7 days to obtain fermenting bacterial suspension of Streptomyces jingyangensis; with determination, viable count of the bacteria in the fermenting bacterial suspension is 30×-50×.

(3) Various components of fermenting bacterial suspension were uniformly mixed in a 1000 L fluid reservoir with a volume ratio of: 10% of Rhodopseudomonas palustris, 10% of Bacillus subtilis, 30% of Saccharomyces cerevisiae, 45% of Lactobacillus acidophilus and 5% of Streptomyces jingyangensis, obtaining 1000 L of first level compound bacteria.

Second Level Complex Fermentation:

2450 L of sterile water was added to 1000 L of the first level compound bacteria to dilute it, then 150 L of Lactobacillus plantarum, 150 L of Lactobacillus acidophilus, 150 L of Saccharomyces cerevisiae and 100 L of brown sugar were added to mix uniformly in a fluid reservoir, fermented under sterile and closed condition, cultured at 30° C. with a pH of 7 for 3 days to obtain 4000 L of second level compound bacteria.

With determination, total count of the viable bacteria in the compound bacteria is 20×-30×. Wherein, there were 8% of Rhodopseudomonas palustris, 12% of Bacillus subtilis, 32% of Saccharomyces cerevisiae, 44% of Lactobacillus acidophilus and 4% of Streptomyces jingyangensis.

### Example 2

### Preparative Method of the Complex Microbial Preparation

First Level Complex Fermentation:

(1) 500 ml of eggplant bottle strains of Rhodopseudomonas palustris was inoculated in 60 L of the first level culture media for Rhodopseudomonas palustris, cultured at 32° C. with a pH of 8 and light intensity of 3500 Lux for 8 days to obtain first level bacteria of Rhodopseudomonas palustris;

500 ml of eggplant bottle strains of Bacillus subtilis was inoculated in 60 L of the first level culture media for Bacillus subtilis, cultured at 32° C. with a pH of 7 for 2 days to obtain first level bacteria of Bacillus subtilis;

500 ml of eggplant bottle strains of Saccharomyces cerevisiae was inoculated in 60 L of the first level culture media for Saccharomyces cerevisiae, cultured at 28° C. with a pH of 5 for 1 days to obtain first level bacteria of Saccharomyces cerevisiae;

500 ml of eggplant bottle strains of Lactobacillus acidophilus was inoculated in 60 L of the first level culture media for Lactobacillus acidophilus, cultured at 32° C. with a pH of 7 for 3 days to obtain first level bacteria of Lactobacillus acidophilus;

500 ml of eggplant bottle strains of Streptomyces jingyangensis was inoculated in 60 L of the first level culture media for Streptomyces jingyangensis, cultured at 30° C. with a pH of 7 for 6 days to obtain first level bacteria of Streptomyces jingyangensis;

(2) 60 L of the first level bacteria of Rhodopseudomonas palustris was inoculated in 600 L of second level culture media for Rhodopseudomonas palustris, cultured by intermittent aerobic cultivation at 32° C. with a pH of 8 and light intensity of 3500 Lux for 8 days to obtain fermenting bacterial suspension of Rhodopseudomonas palustris; with determination, viable count of the bacteria in the fermenting bacterial suspension is 5×-10×.

60 L of the first level bacteria of Bacillus subtilis was inoculated in 600 L of second level culture media for Bacillus subtilis, cultured by aerobic cultivation at 32° C. with a pH of 7 for 2 days to obtain fermenting bacterial suspension of Bacillus subtilis; with determination, viable count of the bacteria in the fermenting bacterial suspension is 30×-50×.

60 L of the first level bacteria of Saccharomyces cerevisiae was inoculated in 600 L of second level culture media for Saccharomyces cerevisiae, cultured by aerobic cultivation at 28° C. with a pH of 5 for 1 days to obtain fermenting bacterial suspension of Saccharomyces cerevisiae; with determination, viable count of the bacteria in the fermenting bacterial suspension is 5×-10×.

60 L of the first level bacteria of Lactobacillus acidophilus was inoculated in 600 L of second level culture media for Lactobacillus acidophilus, cultured by static anaerobic cultivation at 32° C. with a pH of 7 for 3 days to obtain fermenting bacterial suspension of Lactobacillus acidophilus; with determination, viable count of the bacteria in the fermenting bacterial suspension is 20×-30×.

60 L of the first level bacteria of Streptomyces jingyangensis was inoculated in 600 L of second level culture media for Streptomyces jingyangensis, cultured by aerobic cultivation at 30° C. with a pH of 7 for 6 days to obtain fermenting bacterial suspension of Streptomyces jingyangensis; with determination, viable count of the bacteria in the fermenting bacterial suspension is 30×-50×.

(3) Various components of comfermenting bacterial suspension were uniformly mixed in a 1000 L fluid reservoir with a volume ratio of: 5% of Rhodopseudomonas palustris, 14% of Bacillus subtilis, 32% of Saccharomyces cerevisiae, 41% of Lactobacillus acidophilus and 8% of Streptomyces jingyangensis, obtaining 1000 L of first level compound bacteria.

Second Level Complex Fermentation:

2150 L of sterile water was added to 1000 L of the first level compound bacteria to dilute it, then 100 L of Lactobacillus plantarum, 100 L of Lactobacillus acidophilus, 100 L of Saccharomyces cerevisiae and 50 L of brown sugar were added to mix uniformly in a fluid reservoir, fermented under sterile and closed condition, cultured at 25° C. with a pH of 5 for 2 days to obtain 3500 L of second level compound bacteria.

With determination, total count of the viable bacteria in the compound bacteria is 20×-30×. Wherein, there were 6% of Rhodopseudomonas palustris, 15% of Bacillus subtilis, 33% of Saccharomyces cerevisiae, 42% of Lactobacillus acidophilus and 4% of Streptomyces jingyangensis

### Example 3

### Preparative Method of the Complex Microbial Preparation

First Level Complex Fermentation:

(1) 600 ml of eggplant bottle strains of Rhodopseudomonas palustris was inoculated in 60 L of the first level culture media for Rhodopseudomonas palustris, cultured at 36° C. with a pH of 10 and light intensity of 4000 Lux for 7 days to obtain first level bacteria of Rhodopseudomonas palustris;

600 ml of eggplant bottle strains of Bacillus subtilis was inoculated in 60 L of the first level culture media for Bacillus subtilis, cultured at 34° C. with a pH of 8 for 2 days to obtain first level bacteria of Bacillus subtilis;

600 ml of eggplant bottle strains of Saccharomyces cerevisiae was inoculated in 60 L of the first level culture media for Saccharomyces cerevisiae, cultured at 30° C. with a pH of 7 for 1 days to obtain first level bacteria of Saccharomyces cerevisiae;

600 ml of eggplant bottle strains of Lactobacillus acidophilus was inoculated in 60 L of the first level culture media for Lactobacillus acidophilus, cultured at 34° C. with a pH of 8 for 2 days to obtain first level bacteria of Lactobacillus acidophilus;

600 ml of eggplant bottle strains of Streptomyces jingyangensis was inoculated in 60 L of the first level culture media for Streptomyces jingyangensis, cultured at 32° C. with a pH of 8 for 5 days to obtain first level bacteria of Streptomyces jingyangensis;

(2) 60 L of the first level bacteria of Rhodopseudomonas palustris was inoculated in 600 L of second level culture media for Rhodopseudomonas palustris, cultured by intermittent aerobic cultivation at 36° C. with a pH of 10 and light intensity of 4000 Lux for 7 days to obtain fermenting bacterial suspension of Rhodopseudomonas palustris; with determination, viable count of the bacteria in the fermenting bacterial suspension is 5×-10×.

60 L of the first level bacteria of Bacillus subtilis was inoculated in 600 L of second level culture media for Bacillus subtilis, cultured by aerobic cultivation at 34° C. with a pH of 8 for 2 days to obtain fermenting bacterial suspension of Bacillus subtilis; with determination, viable count of the bacteria in the fermenting bacterial suspension is 30×-50×.

60 L of the first level bacteria of Saccharomyces cerevisiae was inoculated in 600 L of second level culture media for Saccharomyces cerevisiae, cultured by aerobic cultivation at 30° C. with a pH of 7 for 1 days to obtain fermenting bacterial suspension of Saccharomyces cerevisiae; with determination, viable count of the bacteria in the fermenting bacterial suspension is 5×-10×.

60 L of the first level bacteria of Lactobacillus acidophilus was inoculated in 600 L of second level culture media for Lactobacillus acidophilus, cultured by static anaerobic cultivation at 34° C. with a pH of 8 for 2 days to obtain fermenting bacterial suspension of Lactobacillus acidophilus; with determination, viable count of the bacteria in the fermenting bacterial suspension is 20×-30×.

60 L of the first level bacteria of Streptomyces jingyangensis was inoculated in 600 L of second level culture media for Streptomyces jingyangensis, cultured by aerobic cultivation at 32° C. with a pH of 8 for 5 days to obtain fermenting bacterial suspension of Streptomyces jingyangensis; with determination, viable count of the bacteria in the fermenting bacterial suspension is 30×-50×.

(3) Various components of comfermenting bacterial suspension were uniformly mixed in a 1000 L fluid reservoir with a volume ratio of: 8% of Rhodopseudomonas palustris, 12% of Bacillus subtilis, 35% of Saccharomyces cerevisiae, 42% of Lactobacillus acidophilus and 3% of Streptomyces jingyangensis, obtaining 1000 L of first level compound bacteria.

Second Level Complex Fermentation:

3250 L of sterile water was added to 1000 L of the first level compound bacteria to dilute it, then 200 L of Lactobacillus plantarum, 200 L of Lactobacillus acidophilus, 200 L of Saccharomyces cerevisiae and 150 L of brown sugar were added to mix uniformly in a fluid reservoir, fermented under sterile and closed condition, cultured at 35° C. with a pH of 8 for 3 days to obtain 5000 L of second level compound bacteria.

With determination, total count of the viable bacteria in the compound bacteria is 20×-30×. Wherein, there were 7% of Rhodopseudomonas palustris, 11% of Bacillus subtilis, 34% of Saccharomyces cerevisiae, 43% of Lactobacillus acidophilus and 5% of Streptomyces jingyangensis

### Example 4

### Preparative Method of the Complex Microbial Preparation

First Level Complex Fermentation:

(1) 600 ml of eggplant bottle strains of Rhodopseudomonas palustris was inoculated in 60 L of the first level culture media for Rhodopseudomonas palustris, cultured at 36° C. with a pH of 10 and light intensity of 4000 Lux for 7 days to obtain first level bacteria of Rhodopseudomonas palustris;

600 ml of eggplant bottle strains of Bacillus subtilis was inoculated in 60 L of the first level culture media for Bacillus subtilis, cultured at 34° C. with a pH of 8 for 2 days to obtain first level bacteria of Bacillus subtilis;

600 ml of eggplant bottle strains of Saccharomyces cerevisiae was inoculated in 60 L of the first level culture media for Saccharomyces cerevisiae, cultured at 30° C. with a pH of 7 for 1 days to obtain first level bacteria of Saccharomyces cerevisiae;

600 ml of eggplant bottle strains of Lactobacillus acidophilus was inoculated in 60 L of the first level culture media for Lactobacillus acidophilus, cultured at 34° C. with a pH of 8 for 2 days to obtain first level bacteria of Lactobacillus acidophilus;

600 ml of eggplant bottle strains of Streptomyces jingyangensis was inoculated in 60 L of the first level culture media for Streptomyces jingyangensis, cultured at 32° C. with a pH of 8 for 5 days to obtain first level bacteria of Streptomyces jingyangensis;

(2) 60 L of the first level bacteria of Rhodopseudomonas palustris was inoculated in 600 L of second level culture media for Rhodopseudomonas palustris, cultured by intermittent aerobic cultivation at 36° C. with a pH of 10 and light intensity of 4000 Lux for 7 days to obtain fermenting bacterial suspension of Rhodopseudomonas palustris; with determination, viable count of the bacteria in the fermenting bacterial suspension is 5×-10×.

60 L of the first level bacteria of Bacillus subtilis was inoculated in 600 L of second level culture media for Bacillus subtilis, cultured by aerobic cultivation at 34° C. with a pH of 8 for 2 days to obtain fermenting bacterial suspension of Bacillus subtilis; with determination, viable count of the bacteria in the fermenting bacterial suspension is 30×-50×.

60 L of the first level bacteria of Saccharomyces cerevisiae was inoculated in 600 L of second level culture media for Saccharomyces cerevisiae, cultured by aerobic cultivation at 30° C. with a pH of 7 for 1 days to obtain fermenting bacterial suspension of Saccharomyces cerevisiae; with determination, viable count of the bacteria in the fermenting bacterial suspension is 5×-10×.

60 L of the first level bacteria of Lactobacillus acidophilus was inoculated in 600 L of second level culture media for Lactobacillus acidophilus, cultured by static anaerobic cultivation at 34° C. with a pH of 8 for 2 days to obtain fermenting bacterial suspension of Lactobacillus acidophilus; with determination, viable count of the bacteria in the fermenting bacterial suspension is 20×-30×.

60 L of the first level bacteria of Streptomyces jingyangensis was inoculated in 600 L of second level culture media for Streptomyces jingyangensis, cultured by aerobic cultivation at 32° C. with a pH of 8 for 5 days to obtain fermenting bacterial suspension of Streptomyces jingyangensis; with determination, viable count of the bacteria in the fermenting bacterial suspension is 30×-50×.

(3) Various components of comfermenting bacterial suspension were uniformly mixed in a 1000 L fluid reservoir with a volume ratio of: 15% of Rhodopseudomonas palustris, 20% of Bacillus subtilis, 20% of Saccharomyces cerevisiae, 35% of Lactobacillus acidophilus and 10% of Streptomyces jingyangensis, obtaining 1000 L of first level compound bacteria.

Second Level Complex Fermentation:

3250 L of sterile water was added to 1000 L of the first level compound bacteria to dilute it, then 200 L of Lactobacillus plantarum, 200 L of Lactobacillus acidophilus, 200 L of Saccharomyces cerevisiae and 150 L of brown sugar were added to mix uniformly in a fluid reservoir, fermented under sterile and closed condition, cultured at 35° C. with a pH of 8 for 3 days to obtain 5000 L of second level compound bacteria.

With determination, total count of the viable bacteria in the compound bacteria is 20×-30×. Wherein, there were 12% of Rhodopseudomonas palustris, 17% of Bacillus subtilis, 25% of Saccharomyces cerevisiae, 38% of Lactobacillus acidophilus and 8% of Streptomyces jingyangensis

### Example 5

### Preparative Method of the Complex Microbial Preparation

First Level Complex Fermentation:

(1) 300 ml of eggplant bottle strains of Rhodopseudomonas palustris was inoculated in 60 L of the first level culture media for Rhodopseudomonas palustris, cultured at 28° C. with a pH of 6 and light intensity of 3000 Lux for 10 days to obtain first level bacteria of Rhodopseudomonas palustris;

300 ml of eggplant bottle strains of Bacillus subtilis was inoculated in 60 L of the first level culture media for Bacillus subtilis, cultured at 28° C. with a pH of 6 for 3 days to obtain first level bacteria of Bacillus subtilis;

300 ml of eggplant bottle strains of Saccharomyces cerevisiae was inoculated in 60 L of the first level culture media for Saccharomyces cerevisiae, cultured at 25° C. with a pH of 4 for 2 days to obtain first level bacteria of Saccharomyces cerevisiae;

300 ml of eggplant bottle strains of Lactobacillus acidophilus was inoculated in 60 L of the first level culture media for Lactobacillus acidophilus, cultured at 28° C. with a pH of 6 for 5 days to obtain first level bacteria of Lactobacillus acidophilus;

300 ml of eggplant bottle strains of Streptomyces jingyangensis was inoculated in 60 L of the first level culture media for Streptomyces jingyangensis, cultured at 27° C. with a pH of 6 for 7 days to obtain first level bacteria of Streptomyces jingyangensis;

(2) 60 L of the first level bacteria of Rhodopseudomonas palustris was inoculated in 600 L of second level culture media for Rhodopseudomonas palustris, cultured by intermittent aerobic cultivation at 28° C. with a pH of 6 and light intensity of 3000 Lux for 10 days to obtain fermenting bacterial suspension of Rhodopseudomonas palustris; with determination, viable count of the bacteria in the fermenting bacterial suspension is 5×-10×.

60 L of the first level bacteria of Bacillus subtilis was inoculated in 600 L of second level culture media for Bacillus subtilis, cultured by aerobic cultivation at 28° C. with a pH of 6 for 3 days to obtain fermenting bacterial suspension of Bacillus subtilis; with determination, viable count of the bacteria in the fermenting bacterial suspension is 30×-50×.

60 L of the first level bacteria of Saccharomyces cerevisiae was inoculated in 600 L of second level culture media for Saccharomyces cerevisiae, cultured by aerobic cultivation at 25° C. with a pH of 4 for 2 days to obtain fermenting bacterial suspension of Saccharomyces cerevisiae; with determination, viable count of the bacteria in the fermenting bacterial suspension is 5×-10×.

60 L of the first level bacteria of Lactobacillus acidophilus was inoculated in 600 L of second level culture media for Lactobacillus acidophilus, cultured by static anaerobic cultivation at 28° C. with a pH of 6 for 5 days to obtain fermenting bacterial suspension of Lactobacillus acidophilus; with determination, viable count of the bacteria in the fermenting bacterial suspension is 20×-30×.

60 L of the first level bacteria of Streptomyces jingyangensis was inoculated in 600 L of second level culture media for Streptomyces jingyangensis, cultured by aerobic cultivation at 27° C. with a pH of 6 for 7 days to obtain fermenting bacterial suspension of Streptomyces jingyangensis; with determination, viable count of the bacteria in the fermenting bacterial suspension is 30×-50×.

(3) Various components of comfermenting bacterial suspension were uniformly mixed in a 1000 L fluid reservoir with a volume ratio of: 15% of Rhodopseudomonas palustris, 20% of Bacillus subtilis, 25% of Saccharomyces cerevisiae, 30% of Lactobacillus acidophilus and 10% of Streptomyces jingyangensis, obtaining 1000 L of first level compound bacteria.

Second Level Complex Fermentation:

2450 L of sterile water was added to 1000 L of the first level compound bacteria to dilute it, then 150 L of Lactobacillus plantarum, 150 L of Lactobacillus acidophilus, 150 L of Saccharomyces cerevisiae and 100 L of brown sugar were added to mix uniformly in a fluid reservoir, fermented under sterile and closed condition, cultured at 30° C. with a pH of 7 for 3 days to obtain 4000 L of second level compound bacteria.

With determination, total count of the viable bacteria in the compound bacteria is 20×-30×. Wherein, there were 13% of Rhodopseudomonas palustris, 16% of Bacillus subtilis, 29% of Saccharomyces cerevisiae, 33% of Lactobacillus acidophilus and 9% of Streptomyces jingyangensis

Experimental example 1 the use of the complex microbial preparation according to the invention in producing medicaments for treatment of diabetes

1. General data: there are 103 out-patients, including 81 males and 22 females; age: 40-70 years old.

2. Diagnostic standards: according to the diagnostic standards established by WHO, most of the above patients were diagnosed as patients with mid-term or long-term diabetes.

3. Therapeutic method: the complex microbial preparation according to the invention produced in Example 1 was orally administered to patients with diabetes three times a day with 20 ml each time in 10 minutes after each meal. 15 days was taken as a course of treatment, 6 courses in total are required.

Notice: 1) shake up the medicine with the bottom up before taking it; 2) don't drink water in 1 hour after taking medicine; 3) don't need to go on a diet; 4) for a patient who are taking insulin, the dosage of the insulin can be reduced gradually in 7 days, and stop to take insulin after the blood glucose level returns to normal; 5) the dosage of other medicines for diabetes can be reduced gradually in 15 days, and stop to take the medicines after the blood glucose level returns to normal; 6) keep in dark place at room temperature.

4. Standards of curative effect evaluation

Significant effect: the level of fast blood glucose and blood glucose in 2 hours after meal are decreased to normal range; or the deceased level of fast blood glucose and blood glucose in 2 hours after meal is over 40% of the level before treatment; the level of glycosylated hemoglobin is decreased to normal range; or the deceased level of glycosylated hemoglobin is over 30% of the level before treatment.

Symptomatic relief: the deceased level of fast blood glucose and blood glucose in 2 hours after meal is over 20% of the level before treatment, but it does not reach the standards of significant effect; the deceased level of glycosylated hemoglobin is over 10% of the level before treatment, but it does not reach the standards of significant effect.

No effect: the level of fast blood glucose and blood glucose in 2 hours after meal are not decreased, or the decreased level does not reach the standards of symptomatic relief; the level of glycosylated hemoglobin is not decreased, or the decreased level does not reach the standards of symptomatic relief.

5. The results of treatment (see: Table 1)

TABLE 1
Curative effect observation on patients treated with the
complex microbial preparation according to the invention

| | Significant effect | | Symptomatic relief | | No effect | | |
|---|---|---|---|---|---|---|---|
| Treatment | | Significant | | Symptomatic | | No effect | Effective |
| duration | Cases | effect rate, % | Cases | relief rate, % | Cases | rate, % | rate, % |
| 15 days | 21 | 20.39 | 59 | 57.28 | 23 | 22.33 | 77.67 |
| 45 days | 47 | 45.63 | 43 | 41.75 | 13 | 12.62 | 87.38 |
| 3 months | 61 | 59.22 | 36 | 34.95 | 6 | 5.83 | 94.17 |

Typical Cases:

Mr. Mao: male, 61 years old; suffering from diabetes for 18 years and taking insulin for 9 years; before taking the complex microbial preparation according to the invention, Mr. Mao went on a diet and was in poor health with 18.5 mmol/L of highest blood glucose level, 7.6 mmol/L of triglyceride and 9.4 mmol/L of total cholesterol. After taking the complex microbial preparation according to the invention for 15 days, Mr. Mao stopped to take insulin and the dosage of other medicines for diabetes were reduced by 50%; then Mr. Mao didn't go on a diet, and the level of blood glucose level, triglyceride and cholesterol were apparently decreased; with detection after 45 days, the health and diet for Mr. Mao were comprehensively returned to normal with 5.7 mmol/L of blood glucose, 2.3 mmol/L of triglyceride and 4.5 mmol/L of total cholesterol.

Mr. Liu: male, 57 years old; suffering from diabetes for 17 years and taking insulin for 6 years; before taking the complex microbial preparation according to the invention, Mr. Liu went on a diet and was in poor health with 16.9 mmol/L of highest blood glucose level, 6.9 mmol/L of triglyceride and 8.2 mmol/L of total cholesterol. After taking the complex microbial preparation according to the invention for 13 days, Mr. Liu stopped to take insulin and the dosage of other medicines for diabetes were reduced by 50%; then Mr. Liu didn't need to go on a diet, and the level of blood glucose level, triglyceride and cholesterol were apparently decreased; with detection after 45 days, the health and diet for Mr. Liu were comprehensively returned to normal with 4.6 mmol/L of blood glucose, 2.8 mmol/L of triglyceride and 5.3 mmol/L of total cholesterol.

The complex microbial preparation according to the invention can rapidly regulate metabolic function of human body, repair and recombine pancreatic islet cell mass, transform the redundant glucose in the blood, reduce the burden of impaired pancreatic 13 cells, gradually recover the function of the impaired pancreas islets, improve microcirculation perfusion, improve the velocity of nerve transmission so as to increase acral temperature, improve sleep of the patients with neurological complication, maintain steady state of the inter environment, keep the balance of organism, correct metabolic disorder and unbalance, can be used for treatment of diabetes and the complications and improving life quality of diabetic patients.

1. A complex microbial preparation, comprising:
5-15% by volume of Photosynthetic bacterium;
10-20% by volume of Bacillus;
20-35% by volume of at least one yeast;
30-45% by volume of Lactobacillus; and
3-10% by volume of Actinomycetes.

2. The complex microbial preparation according to claim 1, wherein the Photosynthetic bacterium comprises 5-10% by volume, the Bacillus comprises 10-14% by volume, the at least one yeast comprises 30-35% by volume, the Lactobacillus comprises 41-45% by volume and the Actinomycetes comprises 3-8% by volume.

3. The complex microbial preparation according to claim 2, wherein the Photosynthetic bacterium comprises 10% by volume, the Bacillus comprises 10% by volume, the at least one yeast comprises 30% by volume, the Lactobacillus comprises 45% by volume and the Actinomycetes comprises 5% by volume.

4. The complex microbial preparation according to claim 1, wherein the Photosynthetic bacterium is Rhodopseudomonas palustris, the Bacillus is Bacillus subtilis, the at least one yeast is Saccharomyces cerevisiae, the Lactobacillus is Lactobacillus acidophilus and the Actinomycetes is Streptomyces jingyangensis.

5. A method of preparing a complex microbial preparation, comprising:
providing 5-15% by volume of Photosynthetic bacterium, 10-20% by volume of Bacillus, 20-35% by volume of at least one yeast, 30-45% by volume of Lactobacillus, and 3-10% by volume of Actinomycetes; and
performing first and second level complex fermentations of the Photosynthetic bacterium, Bacillus, at least one yeast, Lactobacillus and Actinomycetes.

6. The method according to claim 5, wherein the first level complex fermentation comprises:
separately inoculating the Photosynthetic bacterium, Bacillus, at least one yeast, Lactobacillus and Actinomycetes on first level culture media and conducting a first level cultivation to obtain first level bacteria of each component;
separately inoculating each of the obtained first level bacteria on second level culture media and conducting a second level cultivation to obtain fermenting bacterial liquids of each component; and
mixing the obtained fermenting bacterial liquids of each component to obtain first level compound bacteria.

7. The method according to claim 6, further comprising culturing the Photosynthetic bacterium by intermittent aerobic cultivation; culturing the Bacillus by aerobic cultivation, culturing the at least one yeast by aerobic cultivation; culturing the Lactobacillus by static anaerobic cultivation, and culturing the Actinomycetes by aerobic cultivation.

8. The method according to claim 5, wherein performing the second level complex fermentation comprises adding sterile water to the first level compound bacteria to dilute it 2 to 4 times, adding 10-20% of Lactobacillus plantarum, 10-20% of Lactobacillus acidophilus, 10-20% of Saccharomyces cerevisiae and 5-15% of brown sugar thereto, mixing uniformly in a fluid reservoir, fermenting under sterile and closed condition, and culturing with a pH of 5 to 8 at 25-35° C. for 2 to 3 days to obtain second level compound bacteria.

9-10. (canceled)

11. The method of claim 5, further comprising using the complex microbial preparation for the treatment of diabetes.

12. A medicament for the treatment of diabetes, comprising:
a complex microbial preparation, comprised of:
5-15% by volume of Photosynthetic bacterium;
10-20% by volume of Bacillus;
20-35% by volume of at least one yeast;
30-45% by volume of Lactobacillus; and
3-10% by volume of Actinomycetes.

13. The medicament of claim 12, wherein the Photosynthetic bacterium comprises 5-10% by volume, the Bacillus comprises 10-14% by volume, the at least one yeast comprises 30-35% by volume, the Lactobacillus comprises 41-45% by volume and the Actinomycetes comprises 3-8% by volume.

14. The medicament of claim 13, wherein the Photosynthetic bacterium comprises 10% by volume, the Bacillus comprises 10% by volume, the at least one yeast comprises 30% by volume, the Lactobacillus comprises 45% by volume and 5% by volume of the Actinomycetes comprises 5% by volume.

15. The medicament of claim 12, wherein the Photosynthetic bacterium is Rhodopseudomonas palustris, the Bacillus is Bacillus subtilis, the at least one yeast is Saccharomyces cerevisiae, the Lactobacillus is Lactobacillus acidophilus and the Actinomycetes is Streptomyces jingyangensis.
